(19) United States
(12) Patent Application Publication
(10) Pub. No.: US 2025/0073062 A1
(43) Pub. Date: Mar. 6, 2025

(54) APPARATUS, SYSTEM AND METHOD FOR CORNEAL BIOMECHANICS DIAGNOSTICS

(71) Applicant: Alcon Inc., Fribourg (CH)

(72) Inventors: Gangjun LIU, Portland, OR (US); Lingfeng YU, Rancho Santa Margarita, CA (US); Yilin ZHAO, Irvine, CA (US)

(21) Appl. No.: 18/805,420

(22) Filed: Aug. 14, 2024

Related U.S. Application Data
(60) Provisional application No. 63/579,455, filed on Aug. 29, 2023.

(51) Int. Cl. A61F 9/00; A61B 3/10

(52) U.S. Cl. CPC A61F 9/0026; A61B 3/102

(57) ABSTRACT

Described herein include a system and method for a device for drug delivery and ophthalmic diagnostics designed to assist in the diagnosis of the physical state of a cornea through the projection of droplets onto the eye. In some embodiments, the apparatus includes a liquid sampling unit, an electric droplet generator, and a steering unit. In some embodiments, the liquid sampling unit is configured to project a quantity of liquid, while the electric droplet generator receives the quantity of liquid and outputs one or more electrically charged droplets. The steering unit uses electrostatic forces to steer the electrically charged droplets along a trajectory onto the cornea of the patient's eye.

### CROSS-REFERENCE TO RELATED APPLICATIONS

This application claim benefit of and priority to U.S. Provisional Patent Application No. 63/579,455, filed Aug. 29, 2023, which is hereby assigned to the assignee hereof and hereby expressly incorporated by reference in its entirety as if fully set forth below and for all applicable purposes.

### INTRODUCTION

The predictability of corneal response and healing is a key source of error in cataract refractive, corneal refractive, and corneal disease treatment outcomes. Refractive surgery planning uses a population-based average of corneal biomechanics. Yet ˜1% of LASIK patients experience ectasia. Ectasia is a condition characterized by the thinning and bulging of the cornea, which results in an irregular shape of the cornea, leading to progressive myopia (nearsightedness) and astigmatism (distorted vision),

Post-LASIK ectasia is another form of corneal ectasia, which can occur following LASIK surgery, a common form of refractive surgery designed to correct vision. In post-LASIK ectasia, the cornea weakens and bulges forward after the surgery, which can negatively affect vision. The progression of ectasia can lead to significant visual impairment. Early stages might be managed with corrective lenses, but severe cases may require treatments such as corneal cross-linking (a treatment to strengthen the cornea), special contact lenses, or even corneal transplant.

Surgically induced astigmatism (SIA) is a type of vision impairment that can occur as a result of certain types of eye surgery. Astigmatism is a common refractive error of the eye that results in distorted or blurred vision. In SIA, the astigmatism is caused by the changes to the shape of the cornea that occur as a result of the surgical procedure. SIA can occur after a variety of surgical procedures, including LASIK, PRK, and cataract surgery. The exact cause of SIA will depend on the specific surgical procedure, but it is generally related to the way that the cornea is manipulated or reshaped during the procedure.

### SUMMARY

The embodiments described herein provide a method for diagnosis of cornea biomechanics of a patient's eye. In some embodiments, the method includes projecting liquid droplets formed from a quantity of liquid and electrically charging the liquid droplets to form electrically charged droplets. The electrically charged droplets are steered along a trajectory onto the cornea using a dynamic electric field. In some embodiments, the method includes measuring, using an OCT measurement unit, the response to an impact of the electrically charged droplets on the cornea. In some embodiments, the measured response is used to determine physical parameters of the cornea (i.e., corneal biomechanics). The physical parameters of the cornea are used to determine the condition of the cornea. In some embodiments, the diagnosis is output based on the condition of the cornea.

Some embodiments include an apparatus for projecting droplets onto a cornea of a patient's eye for measuring corneal biomechanics used in ophthalmic diagnostics. Some embodiments include a liquid sampling unit, an electric droplet generator, and a steering unit. In some embodiments, the liquid sampling unit is configured to project a quantity of liquid. In some embodiments, the electric droplet generator is configured to receive the quantity of liquid and output one or more electrically charged droplets. In some embodiments, the steering unit is configured to electrostatically steer the one or more electrically charged droplets along a trajectory onto the cornea of the patient's eye.

In some embodiments, the liquid sampling unit comprises one or more pressurized containers with an outlet and a valve coupled to the outlet via one or more conduits. In some embodiments, the actuation of the valve outputs the quantity of liquid. In some embodiments, the liquid sampling unit includes one or more vented containers with a first outlet, a valve coupled to the first outlet via one or more conduits, a pump coupled to the valve, and a pressure sensor controller coupled to the pump. In some embodiments, the pressure sensor controller is configured to sense the pressure of the quantity of liquid and adjust a flow rate of the pump based on the sensed pressure.

### BRIEF DESCRIPTION OF THE DRAWINGS

FIG. 1 depicts a schematic of an optical diagnostic system, in accordance with some embodiments.

FIGS. 2A-2B depict schematics of a liquid jet-drop apparatus, in accordance with some embodiments.

FIG. 3 depicts a flow chart depicting a method of optical diagnosis, in accordance with some embodiments.

FIG. 4 depicts a schematic of an optical diagnostic system, in accordance with some embodiments.

### DETAILED DESCRIPTION

The present disclosure will now be described in detail with reference to the drawings, which are provided as illustrative examples of the disclosure so as to enable those skilled in the art to practice the disclosure. Notably, the figures and examples below are not meant to limit the scope of the present disclosure to a single embodiment, but other embodiments are possible by way of interchange of some or all of the described or illustrated elements. Moreover, where certain elements of the present disclosure may be partially or fully implemented using known components, only those portions of such known components that are necessary for an understanding of the present disclosure will be described, and detailed descriptions of other portions of such known components will be omitted so as not to obscure the disclosure.

As used herein, the singular form of “a”, “an”, and “the” include plural references unless the context clearly dictates otherwise. As used herein, the statement that two or more parts or components are “coupled” shall mean that the parts are joined or operate together either directly or indirectly (i.e., through one or more intermediate parts or components, so long as a link occurs). As used herein, “directly coupled” means that two elements are directly in contact with each other. As used herein, “fixedly coupled” or “fixed” means that two components are coupled so as to move as one while maintaining a constant orientation relative to each other. As used herein, “operatively coupled” means that two elements are coupled in such a way that the two elements function together. It is to be understood that two elements “operatively coupled” does not require a direct connection or a permanent connection between them. As utilized herein, “substantially” means that any difference is negligible, such that any difference is within an operating tolerance that is known to persons of ordinary skill in the art and provides for the desired performance and outcomes as described in the embodiments described herein. Descriptions of numerical ranges are endpoints inclusive.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality). Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

In the exemplary embodiments described herein, an embodiment showing a singular component should not be considered limiting; the disclosure is intended to encompass other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present disclosure encompasses present and future known equivalents to the known components referred to herein by way of illustration.

Corneal biomechanics, such as corneal stiffness, can play an important role in understanding, diagnosing, and treating diseases such as glaucoma, keratoconus, and ectasia. Detailed clinical assessment of corneal biomechanics has the potential to revolutionize the ophthalmic industry by providing, for example, personalized LASIK (Laser-Assisted in Situ Keratomileusis) and cataract surgery. One way to measure corneal biomechanics is by capturing a corneal response to external forces. For example, to measure corneal stiffness, some devices may apply a high-pressure air pulse to induce a large corneal displacement (e.g., greater than 2 mm). However, the large corneal displacement typically has a significant nonlinear component that may prevent the device from accurately measuring corneal stiffness. In addition, a deformation amplitude of the cornea is affected by the stability of a source of the air pulse.

The ophthalmic diagnostic systems and methods described herein offer a range of advantages for corneal biomechanics diagnostics. Corneal biomechanics diagnostics refers to measuring or quantifying corneal biomechanical properties. Effectively quantifying corneal biomechanical properties of both healthy and diseased corneas contributes to increased predictability and surgeon confidence in providing treatments. For example, as described in detail below, utilizing individualized measurements of the corneal biomechanical properties, provided per the embodiments described herein, improves the ability to predict the risks of surgical intervention of corneas, such as post-LASIK ectasia and improve predictability of corneal treatments. Individualized corneal biomechanics measurements may further be used to evaluate therapeutic interventions with cross-linking, and also monitor the progression/retardation of collagen degradation. Furthermore, because corneal biomechanics are highly correlated with myopia and affect orthokeratology success, individualized corneal biomechanics measurements may provide more accurate myopia diagnosis, which is advantageous because high myopia may increase the risk of glaucoma.

Further, some embodiments herein provide methods and systems for drug delivery during corneal biomechanics diagnostics. Drug delivery, herein, refers to administering liquid droplets containing medicaments for the eye. For example, such medicaments may include viscoelastic agents that keep the cornea hydrated and maintain the corneal shape during OCT measurements and corneal evaluation. In some embodiments, liquid medicaments may include saline solution for rinsing the cornea and internal system components used during OCT measurements and corneal evaluation to remove any debris or bacteria. In some embodiments, liquid medicaments may include anesthetic agents that numb the eye before evaluation to minimize any discomfort or pain to the patient.

Generating accurate and individualized measurements of corneal biomechanical properties, as described herein, further advantageously addresses the existing lack of predictability associated with the post-operative corneal response and healing, a significant source of error in cataract refractive, corneal refractive, and corneal disease treatment outcomes. For example, utilizing the corneal biomechanics diagnostics systems and methods described herein results in enhanced cataract outcomes through patient-specific SIA calculations, better Limbal Relaxing Incision (LRI) outcomes from patient-specific calculations, more informed treatment decisions for corneal refractive procedures, and improved Orthokeratology (Ortho-k) outcomes by generating accurate and individualized measurements of corneal biomechanical properties and tailoring treatments to individual patients, based on such accurate and individualized measurements, based on individual corneal biomechanics mapping.

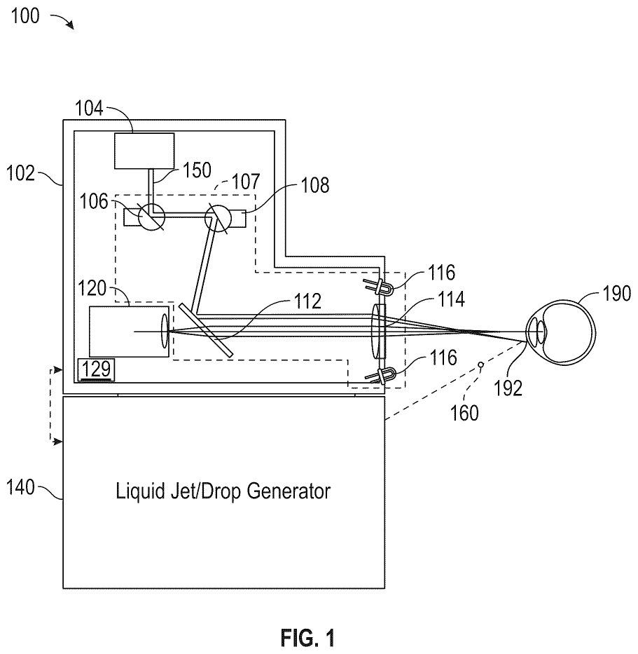

Referring now to FIG. 1, FIG. 1 depicts a corneal biomechanical diagnostic system 100. System 100 includes OCT unit 102 and liquid jet-drop generator 140. Liquid jet-drop generator 140 ejects projectile droplets 160 onto cornea 192 of a patient's eye 190. When a projectile droplet 160 impacts onto cornea 192, the impact causes an excitation of the corneal tissue via the transfer of kinetic energy from projectile droplet 160 onto the tissue of cornea 192. The excitation, or transfer of kinetic energy, causes a visco-elastic and optical response in the cornca including generation of a propagating sheer wave, which are observed by OCT unit 102. As discussed in further detail below, the visco-elastic response of the cornea is a measure of oscillation of the cornea. The optical response includes changes to the shape of the cornea under mechanical stress (e.g., an impact of a liquid projectile) and retro-scattering properties of the cornea. OCT unit 102 is designed to observe visco-elastic and optical responses and generate corresponding data indicative of such responses.

Retro-scattering properties of cornea 192 refer to the phenomenon where light incident on the corneal tissue scatters backward (i.e., backscattering), or in the direction from which it came. The backscattered light can provide information about the optical response of the cornea under mechanical stress, the corneal shape, corneal structure, corneal thickness, curvature, and biomechanical properties. Thus, by analyzing the retro-scattering properties of the cornea, researchers and clinicians can gather valuable information about the cornea shape, structure, and biomechanical properties under various conditions. Retro-scattering can be seen on images of cornea 192 provided by OCT unit 102. Measurements of the retro-scattering on the images may be utilized to visualize and/or map the corneal layers in cornea 192 and assess the shape and structural changes under mechanical stress.

In some embodiments, OCT unit 102 measures mechanical shear wave propagation at the surface of cornea 192. Wave propagation happens when a projectile (e.g., projectile droplet 160) impacts eye 190. As a result of the impact, kinetic energy transfers from the liquid droplet to the surface of the cornea. The transfer of kinetic energy causes a shear wave propagation along the surface of the cornea. As described in further detail below, observing shear wave propagation provides Young's modulus, which may then be utilized for diagnosis. In addition to determining corneal conditions of the eye, having personalized shear wave propagation data for individual patients advantageously provides increased accuracy in pre-operative healing predictions and minimizes the need for post-operative corrections.

As shown in FIG. 1, OCT unit 102 includes OCT interferometer 104, optical components 107, eye-tracking unit 120, and controller 124. The OCT unit 102 operates to produce high-resolution images and/or measurements of eye 190, which provide detailed information about the cornea and other structures within eye 190.

In some embodiments, OCT interferometer 104 emits a laser light beam 150 to generate interference patterns from the light that is reflected from eye 190. Corneal biomechanics data are then derived from high-resolution interference pattern images of eye 190, which provide detailed information about cornea 192 and other structures within eye 190.

In some embodiments, optical components 107 may include fast-scanning mirror 106, slow scanning mirror 108, hot mirror 112, LEDs 116, front-eye optics 114, and/or controller 124. Fast-scanning mirror 106 and slow-scanning mirror 108 operate together to scan laser light beam 150 and obtain three dimensional images of the ocular tissue (not shown). For example, fast-scanning mirror 106 may rapidly scan laser light beam 150 in one direction, while slow-scanning mirror 108 may scan laser light beam 150 in a different direction (e.g., a perpendicular direction). Combining scanning mirrors as described above allows OCT unit 102 to produce high-resolution images of eye 190 in a relatively short amount of time.

In some embodiments, optical components 107 condition and/or modify laser light beam 150 by adjusting one or more parameters of laser light beam 150 (e.g., spot size, beam intensity, and the like). In some embodiments, optical components 107 may include one or more of a beam splitter, beam expanders, and/or a polarizing beam splitter. For example, in some embodiments, a beam splitter splits a beam of light into two or more beams. The beam splitter may be used to split laser light beam 150 and into two separate beams and then recombine, for example, by aligning the peaks and troughs of each beam to create a single, stronger beam. Beam expanders may be used to increase or decrease the spot size of laser light beam 150.

In some embodiments, optical components 107 may include additional beam steering optics (not shown in FIG. 1) for scanning laser light beam 150 along cornea 192. For example, in some embodiments, optical components 107 may include mirrors, prisms, and/or focusing lenses, such as a collimator and/or an objective lens. In some embodiments, optical components 107 may include an optics and alignment mount (not shown) for aligning laser light beams 150 in a precise and stable manner. The optics and alignment mount allow controller 124 to adjust the position and angle of each optical component (e.g., 106, 108, 110, 112, and/or 114) with high precision for conditioning laser light beam 150. In some embodiments, the optics and alignment mount may be incorporated as part of eye-tracking unit 120.

In some embodiments, front-eye optics 114 (e.g., one or more eyepieces) focus laser light beam 150 onto cornea 192. In some embodiments, hot mirror 112 may be used to combine visible light with the near infrared OCT light. In some embodiments, LEDs 116 may be used for the illumination. In some embodiments, front-eye optics 114 may collimate laser light beam 150 and direct laser light beam 150 towards eye 190.

Eye-tracking unit 120 may track the position of eye 190 and maintain a stable connection between laser light beam 150 and eye 190. For example, eye-tracking unit 120 tracks the position of eye 190 and ensures that laser light beam 150 remains accurately focused on cornea 192. Accurately focusing the laser light beam 150 is advantageous because even small movements of eye 190 may cause laser light beam 150 to move away from targeted location on the cornea 192, leading to inaccurate OCT measurements.

In some embodiments, eye-tracking unit 120 uses a combination of cameras (not shown) and algorithms to monitor the position of the eye. Eye monitoring algorithms and operations may be stored in a processor and memory (not shown) of eye-tracking unit 120. The cameras detect the position of the pupil and the position of any reflections from cornea 192. The algorithms then use pupil and reflection position to calculate the position of the eye and to determine adjustments to laser light beam 150.

In some embodiments, eye-tracking unit 120 is in wireless or wired communication with controller 124, which receives data from the eye-tracking unit 120 and uses real-time eye-tracking data to adjust the position of laser light beam 150. Real-time eye tracking allows OCT unit 102 to maintain a stable connection between laser light beam 150 and cornea 192, even when eye 190 moves during the operation of OCT unit 102. For example, to implement real-time eye-tracking, eye-tracking unit 120 uses compensation algorithms to correct for any movement of eye 190 that may occur during the corneal biomechanics evaluation process of the embodiments described herein. Compensation algorithms ensure that OCT unit 102 produces accurate and reliable measurements, even in cases where eye 190 moves during measurement. Compensation algorithms may reside on controller 124 and/or eye-tracking unit 120. Eye-tracking unit 120 is in communication with, and may be controlled by, controller 124.

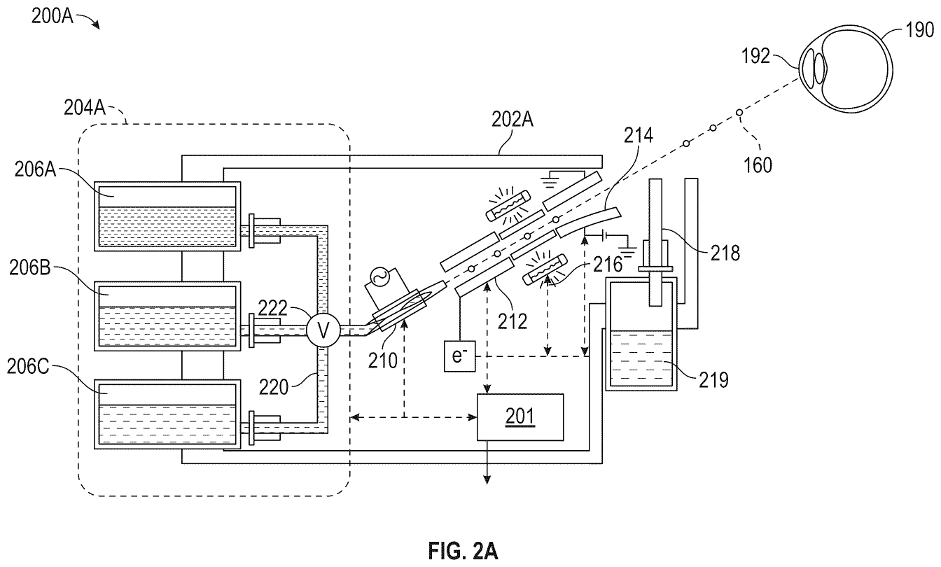

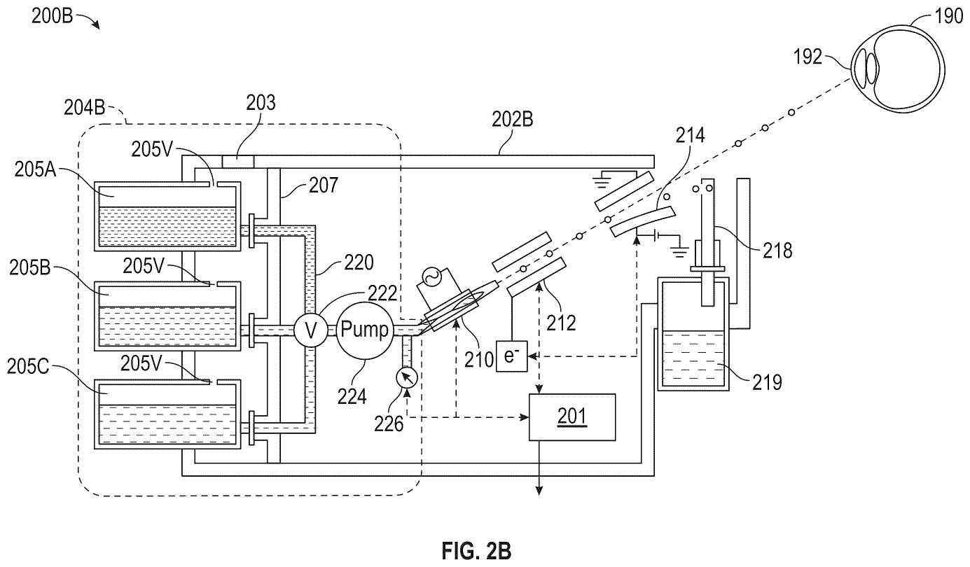

Controller 124 may control the movement of optical components 107 including the scanning of fast-scanning mirror 106 and slow-scanning mirror 108 described above. Controller 124 adjusts the position of laser light beam 150 based on the received data from eye-tracking unit 120. In some embodiments, controlling the movement of optical components 107 and adjusting the position of laser light beam 150 based on the received data from eye-tracking unit 120 may be implemented by controller 201, which is discussed in further detail below (see FIG. 2, infra). Controller 124 includes a processor and memory (not shown) for storing instructions and executing the operations and procedures as described in one or more embodiments herein. As shown in FIG. 1, liquid jet-drop generator 140 is in communication with OCT 102 via controller 124.

In some embodiments, system 100 may utilize different protocols for wireless and/or wired communication between OCT unit 102 and liquid jet-drop generator 140 to ensure reliable and efficient data transfer. One or more wireless communication protocols may be implemented, such as Wi-Fi (IEEE 802.11a/b/g/n/ac/ax), Bluetooth, Bluetooth Low Energy, ZigBee, Z-Wave, and/or other wireless communication protocols that provide secure, low-power, and high-speed communication facilitating seamless data exchange and control in the system 100. In some embodiments, wired communication protocols may be used due to environmental factors, signal interference, or other constraints. In some embodiments, various components of system 100 may be configured to support both wireless and wired communication protocols simultaneously or selectively, depending on the specific requirements.

In some embodiments, LEDs (light emitting diodes) 116 may be used for illumination for the pupil tracking purpose, including for the detection of Purkinje spots. Purkinje spots are reflections of light that occur within the eye and can provide important information about the eye's optical properties and allow for accurate and reliable measurements to be made. The light from the LEDs 116 is directed towards the eye and reflects off of the various structures within the eye, including the Purkinje spots. The reflected light is then captured by cameras of the eye-tracking unit 120 and used to produce images of the eye and to detect the position of the pupil and Purkinje spots. In some embodiments, the LEDs 116 can also be adjusted to produce optimized wavelengths, brightness and consistency of light, depending on the specific requirements.

Referring now to FIGS. 2A-2B, in conjunction with FIG. 1, FIGS. 2A-2B depict examples of liquid jet-drop generator 140, in accordance with some embodiments. In particular, FIGS. 2A-2B depict liquid jet-drop generators 240A, 240B. Liquid jet-drop generator 240A, 240B are embodiments of liquid jet-drop generator 140, wherein similarly labeled parts and numbers correspond to similar features having similar functionality.

In some embodiments, liquid jet-drop generators 240A, 240B include liquid sampling units 204A, 204B, respectively. Liquid sampling unit 204B may generally operate similarly as liquid sampling unit 204A, with some differences, as described in detail below. For example, liquid sampling unit 204B includes vented, non-pressurized storage tanks having vents, wherein a pump and valve provide fluid flow to transfer liquid. In contrast, liquid sampling unit 204A includes pressurized storage tanks, which is described in further detail below.

As shown in FIG. 2A, in some embodiments, liquid jet-drop generator 240A includes housing 202A, liquid sampling unit 204A, piezo-electric actuator 210, electrostatic charging unit 212, electrostatic steering unit 214, ultra-violet (UV) light emitter 216, liquid waste collector 218, and/or waste container 219. To facilitate various types of liquid projectile droplets, in some embodiments, liquid sampling unit 204A includes three pressurized storage tanks 206A, 206B and 206C. In some embodiments, tanks 206A, 206B, 206C contain different types of liquids, or one or more of the same type of liquid, which are used for various functions relating to the evaluation of biomechanical properties of the eye, drug delivery, and sanitation purposes. Tanks 206A-206C each include an outlet (e.g., a threaded gasket coupling) that couples to a conduit 220 for delivering liquid to a piezo-electric actuator 210 when valve 222 is actuated. In some embodiments, tanks 206A-206C maintain the pressure and stability of the liquid medicaments, physiological fluid, and/or rinsing liquid, respectively, ensuring that liquid is delivered in a consistent and accurate manner.

For example, in some embodiments, tank 206A stores liquid medicaments that will be delivered to the eye during, before, or after the diagnostic process. The volume of individual droplets and/or the liquid may be determined based on a dosage of medicament that is to be administered. For example, in some embodiments, the medicament may include the drug SYSTANE® Ultra Lubricant Eye Drop with a volume of 0.05 ml each time.

Tank 206B stores a standard physiological fluid, which may be used for excitation of the corneal tissue during corneal biomechanical evaluation. In some embodiments such physiological fluid or reference liquid may include, for example, Tears NATURALE® Free by ALCON®. In some embodiments, the reference liquid may be administered at a suggested volume of 0.05 ml per administration.

In some embodiments, tank 206C stores a rinsing liquid for sanitizing and/or flushing liquid jet-drop generator 240A between different diagnostic procedures. In some embodiments, a rinsing liquid may include distilled water, which may be administered at 5 ml per administration. In some embodiments, one or more tanks may include the same type of liquid. In some embodiments, liquid sampling unit 204A may include more, or less, than three tanks, for example one tank or four tanks.

In some embodiments, outputting liquid by liquid sampling unit 204A comprises actuating (i.e., opening or closing) valves 222. Actuating valves 222 causes the liquid to flow to piezo-electric actuator 210 via conduits 220. In some embodiments, conduits 220 may include medical grade plastic tubing and/or other suitable material. In some embodiments, valve 222 may include a one-directional valve with feedback protection. Actuation of valve 222 may be performed by controller 201.

In certain embodiments, because liquid sampling unit 204A is pressurized and the liquids are fully contained within pressurized tanks 206A-206C, storage tanks 206A-206C are not required to be isolated from electrical components (e.g., 210, 212, and 214) of liquid jet-drop generator 240A. Although, in some embodiments, electrical components (e.g., 210, 212, 214, and/or 216) may be isolated from storage tanks 206A-206C.

As mentioned above, piezo-electric actuator 210 forms and outputs liquid droplets. Liquid droplets may be formed in various ways. In some embodiments, piezo-electric actuator 210 may oscillate the width of the pathway of the liquid droplets between a wide width and a narrow width. The pathway of the liquid droplets may be formed by a conduit of flexible tubing. For example, by applying a dynamic electrical voltage to piezo-electric actuator 210, the surface of piezo-electric actuator 210 rapidly expands and contracts, thereby oscillating the pathway conduit width. Liquid passing through when the pathway is narrow causes liquid droplet formation due to the tensile strength of the liquid.

In certain other embodiments, piezo-electric actuator 210 may form droplets of liquid by rapidly changing the pressure of the liquid. The rapid expansion and contraction of the surface of piezo-electric actuator may generate increases in air pressure, which acts to separate liquid into individual droplets. Piezo-electric actuator 210 may be coupled to electrostatic charging unit 212.

Electrostatic charging unit 212 is used to charge the surface of liquid droplets with electric charge. Electric charge is induced by applying a high voltage to electrostatic charging unit 212, which then emits an electric field across the pathway of the individual droplets, thereby forming electrically charged droplets. Electric charge in the droplets enables the steering of liquid droplets, discussed in further detail below. Moreover, because the droplets are charged and repel each other, this advantageously reduces the risk of droplets merging and forming larger droplets that are more difficult to control.

In some embodiments, electrostatic charging unit 212 includes a charging electrode and a ground electrode. The charging electrode and ground electrode may be placed perpendicular to the flow direction in a narrow channel for electrostatic droplet charging. When a droplet is forced through or squeezed in the narrow channel, a positive or negative pulse will be applied to the charging electrode to induce the charge.

In some embodiments, electrostatic steering unit 214 controls the trajectory of electrically charged droplets by generating a dynamic electric field. By altering the strength and/or polarity of the electric field, the electrically charged droplets experience a lateral force that causes the electrically charged droplets to move in the direction of the repelling or attracting lateral electric force. For example, electrostatic steering unit 214 may apply a voltage to electrodes having electrostatic plates positioned in close proximity to the charged droplets. Voltage applied to electrodes of electrostatic steering unit 214 creates an electric field that may be used to steer electrically charged droplets in the desired direction. The strength and direction of the electric field may be adjusted to provide fine control over the movement of the charged liquid droplets.

In some embodiments, piezo-electric actuator 210, electrostatic charging unit 212, and electrostatic steering unit 214 may include a variety of electronic components (not shown) that control the movement of charged droplets (e.g., electrodes, power supply, insulators, amplifiers, and/or feedback and control logic). In some embodiments, the power supply voltage can range from a few volts to several kilovolts, depending on the size of liquid droplets. One or more amplifiers may be implemented to control the voltage applied to the electrodes. Amplifiers may be utilized for adjusting the strength and direction of the electric field in real-time, allowing for precise control over the movement of the charged droplets. Feedback and control circuits may monitor the movement of the charged droplets and adjust the electric field accordingly and may include one or more sensors, signal processing circuits, and digital control systems. Other components such as capacitors, resistors, and inductors, may also be implemented in various embodiments, depending on the specific design requirements, the discussion of which is omitted for brevity.

While being steered by electrostatic steering unit 214, in some embodiments of FIG. 2A, an ultraviolet light emitter 216 may be used to project one or more beams of ultraviolet light for sterilizing the electrically charged droplets. Illuminating the individual droplets with UV light sterilizes electrically charged droplets prior to being steered onto the patient's eye via electrostatic steering unit 214. The ultra-violet (UV) light is used to sterilize the droplets, helping to prevent the growth of bacteria and other contaminants that may infect patient's eye 190. In FIG. 2A, UV light emitter is shown as being positioned after the electrostatic charging unit 212. However, in some embodiments, UV light emitter 216 may be positioned at a different location.

In some embodiments, liquid waste collector 218 is coupled to an exterior of housing 202. Liquid waste collector 218 may be utilized for collecting any liquid that is not used in the droplet generation process. Liquid waste container 219 may be moveable and allows for the easy disposal of the collected waste. Liquid waste collector 218 traps errant droplets that fall outside of intended trajectory onto cornea 192. Trapping errant droplets advantageously allows for collection and reuse of costly medicaments that may otherwise go to waste.

Referring now to FIG. 2B, in some embodiments, liquid sampling unit 204B includes vented tanks 205A, 205B, 205C cach having an outlet coupled to inlets of valve 222 via conduits 220. Valve 222 includes an outlet that is coupled to an inlet of pump 224. Tanks 205A-205C are vented to allow for the liquid to be pumped out and projected onto eye 190 through the use of pump 224 and pressure controller 226. Because tanks 205-A-205C are vented, electrical components (e.g., 210, 212, 214, and/or 216) are isolated from storage tanks 206A-206C. Thus, in some embodiments, barrier 207 may provide a physical separation for isolating tanks 205A-205C from electrical components (e.g., 210, 212, 214, and/or 216), as described further below.

Pump 224 includes an outlet that outputs liquid to piezo-electric actuator 210. Pump 224 and pressure controller 226 regulate the flow of each liquid contained by tanks 205. Pressure (e.g., an eyepiece) controller 226 includes a pressure sensor (not shown) that senses the pressure of liquid flowing from the outlet of pump 224 and adjusts the amount of liquid flow in pump 224 based on the sensed pressure of the liquid flowing at the output of pump 224. Pressure controller 226 may be in communication with controller 201 and ensures that the correct liquid is delivered to eye 190 during the corneal biomechanics diagnostics, which is described in detail further below.

Similar to tank 206A, tank 205A may be used for storing a liquid medicament that will be projected onto eye 190 during, before, or after the diagnostic processes, similar to or the same as liquid medicaments discussed above in FIG. 2A. Similar to tank 206B, tank 205B stores a standard physiological fluid, which may be used for excitation of the corneal tissue during biomechanical response observations, which is discussed in detail below. Further, similar to tank 206C, tank 205C stores a rinsing liquid, which may be used to clean or flush liquid jet-drop generator 240B between different diagnostic procedures. Valve 222 and pump 224 control the flow of liquid from each of tank 205A, 205B, 205C, ensuring that the correct liquid is delivered to the eye during the diagnostic process.

As shown in FIG. 2B, liquid sampling unit 204B may be separate from housing 202B via barrier 207. Barrier 207 functions by preventing any contamination or mixing of the different liquids within liquid jet-drop generator 240B. Conduits 220 run through barrier 207, linking each storage tank 205A-205C to valve 222 and pump 224.

In some embodiments, housing 202A, 202B may be constructed from a variety of materials, depending on the specific requirements and design constraints. Material of housing 202A, 202B depends on factors such as mechanical protection, electrical insulation, and/or regulatory requirements. In some embodiments, housing 202A, 202B may be made of a material that provides mechanical protection to liquid sampling unit 204A, 204B components (e.g., 204, 210, 212, 214, 216, and 218). For example, metal alloys, polymers, or composites that are strong and durable. In some embodiments, housing 202A, 202B provides electrical insulation and prevents the buildup of static charge. Accordingly, housing 202A, 202B may include plastic or ceramics. In some embodiments, the material chosen for the housing 202A, 202B may also need to meet specific regulatory requirements, such as those related to biocompatibility or sterilization. Accordingly, stainless steel or medical-grade plastics may be implemented in some embodiments.

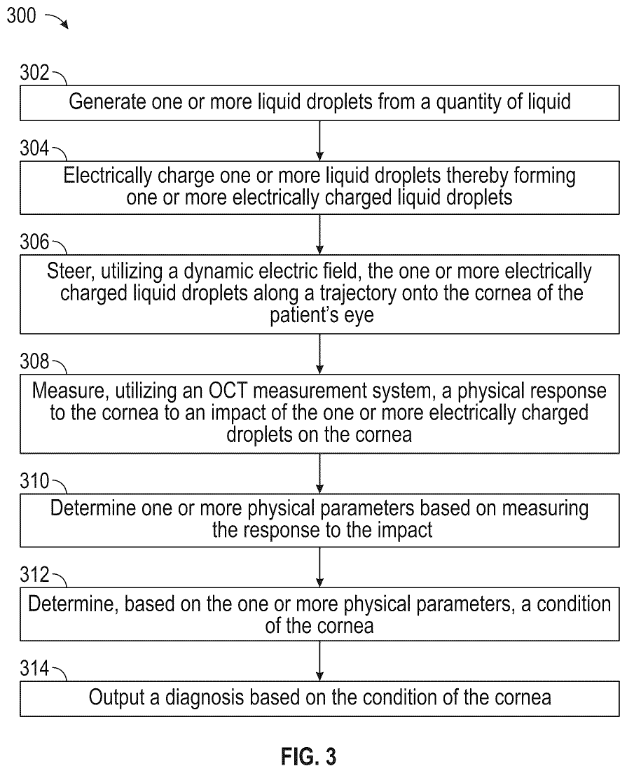

Referring now to FIG. 3 in conjunction with FIGS. 1-2, FIG. 3 depicts method 300, implemented by system 100. Method 300 relates to the evaluation of corneal biomechanical properties by observing the optical and biomechanical response of the cornea caused by the impact of a liquid droplet onto the cornea (e.g., 192) of a patient's eye (e.g., 190). In some embodiments, method 300 begins at step 302 by generating one or more liquid droplets from a liquid. The liquid may be stored and dispensed by liquid sampling unit 204A, 204B. Forming the liquid droplets may be performed by piezo-electric actuator 210, as discussed above.

The method continues at step 304 by electrically charging the liquid droplets, thereby forming electrically charged droplets. Electrically charging the liquid droplets may be performed by electrostatic charging unit 212. The method continues at step 306 by steering the one or more electrically charged droplets utilizing a dynamic electric field along a trajectory onto the cornea of the patient's eye. Electrostatic steering unit 214 may perform step 306.

The method continues, at step 308, by measuring a response to the impact of the electrically charged droplets on cornea 192 utilizing OCT unit 102, as described above. As a result of the impact of the electrically charged droplets, cornea 192 will vibrate and generate shear waves. In some embodiments, measuring the response may include the measurement of the vibration properties of cornea 192 by OCT unit 102. Such vibration properties may include vibration amplitude and vibration frequency. In some embodiments, measuring the response may include measuring mechanical shear wave propagation along the surface of cornea 192 by mean phase-sensitive techniques, which is described in detail below. Propagating shear waves are generated when liquid jet-drops strike the surface of cornea 192 and the wave propagation speed/velocity is related to the corneal biomechanics. Shear waves are mechanical waves that propagate perpendicular to the direction of the applied force (e.g., impact of liquid droplets), causing the particles within a medium (e.g., surface of the cornea) to move parallel to the wavefront.

In some embodiments, phase-sensitive techniques may focus on detecting and analyzing the phase shifts experienced by the shear waves propagating through cornea 192. Such phase information can provide valuable insights into the properties of cornea 192 or the interaction between the wave and cornea 192. For example, in a propagating wave, the phase represents the position of a point within the wave's oscillation cycle at a particular time. The phase can be described by an angle, usually in radians or degrees, and the phase changes as the wave propagates through cornea 192. Phase-sensitive techniques monitor these phase changes and extract information about properties of cornea 192, such as its elasticity, dispersion, or refractive index. Thus, in some embodiments, measuring mechanical shear wave propagation using phase-sensitive techniques may include tracking the movement of shear waves generated within the corneal tissue and detecting the phase shifts in kinetic waves propagating through cornea 192, allowing for assessment of the elastic properties of cornea 192, such as young's modulus. The shear wave speed is directly related to the tissue's elastic modulus, which facilitates the assessment of corneal stiffness.

The method continues at step 310 by determining, utilizing OCT unit 102, one or more physical parameters of the cornea based on the measured response. Physical parameters of the cornea may include response time, vibration amplitude, natural frequency, wave velocity, and the like. At step 312, a condition of the cornea is determined based on the one or more physical parameters. The condition of the cornea may correspond to a Young's Modulus value, topology of the cornea, and/or an intraocular pressure (IOP).

At step 314, the method continues by outputting a diagnosis based on the condition of the cornea and clinical test data. For example, clinical test data may correlate various conditions of the eye with IOP and Young's modulus values and algorithms for characterization of the physical parameters of the cornea and conversion of Young's modulus and IOP. Such data may be stored remotely or as part of system 100 storage (e.g., 416) In some embodiments, the diagnosis in may include cataract treatment based on surgically induced astigmatism, cataract refractive treatment, cataract diffractive treatment, corneal refractive treatment, a risk level of post-LASIK complications, and/or orthokeratology treatment. For example, based on a level of IOP and a known quantity for the Young's modulus, various abnormalities of the cornea may be determined based on correlating the IOP and Young's modulus values to the clinical test data. The condition may be displayed via a user interface of system 100, as discussed further below, or communicated to a user in some other fashion.

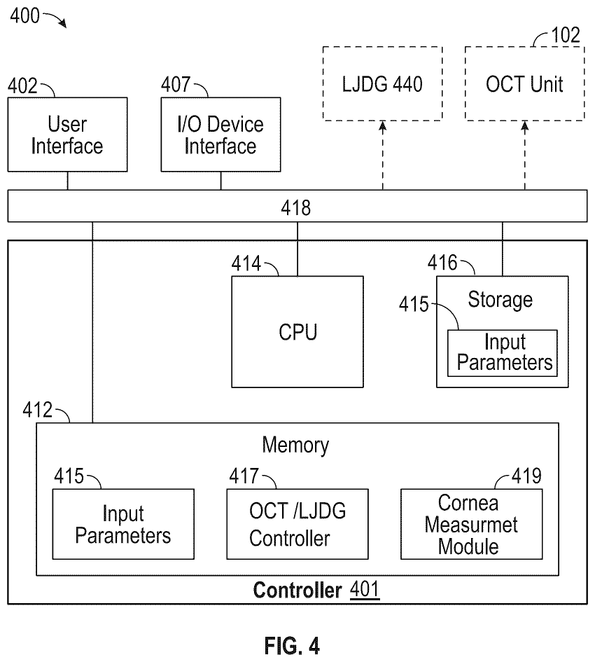

Referring now to FIG. 4, in conjunction with FIGS. 1-3, FIG. 4 depicts a schematic of an example system 400 having controller 401 (e.g., similar to or the same as controller 201) implemented for measuring corneal biomechanical properties of the cornea of a patient's eye, in accordance with some embodiments. As shown in FIG. 4, in some embodiments, controller 401 is in communication with liquid jet-drop generator 440 and OCT unit 102. For example, controller 401 may be in communication with liquid jet-drop generator (LDJG) 440 and/or OCT unit 102 through a network (e.g., wired or wireless network) or a bus 418. Liquid jet-drop generator 440 is an example of liquid jet-drop generator 140, 240A, 240B. In some embodiments, controller 401 may be in wired or wireless communication with liquid jet-drop generator 440 and OCT unit 102. For example, in some embodiments, controller 401 may be part of a system, device, or console that is separate from LJDG 440 and OCT unit 102. In another example, controller 401, LJDG 440, and OCT unit 102 may be housed by the same system, device, or console. In yet other examples, controller 401 may be housed by or part of LJDG 440 (e.g., controller 401 may be the same as controller 124) or be housed by or part of OCT unit 102.

As shown, the controller 401 includes memory 412, processor 414 and storage 416 and is in communication (e.g., through a bus 418) with user interface 402 and I/O device interface 407. Memory 412 includes machine readable instructions for executing one or more embodiment described herein. Processor 414 is in communication with memory 412 and configured to execute the machine-readable instructions stored in memory 412. Processor 414 may include a central processing unit (CPU), a memory, cache, and/or support circuits. In some embodiments, processor 414 may correspond to a single CPU, multiple CPUs, or single CPU having multiple processing cores. Processor 414 may be a general-purpose computer processor configured for use in an ophthalmic setting for controlling LDJG 440 and/or OCT unit 102. Memory 412 may include random access memory, read-only memory, hard disk drive, non-volatile memory, such as a disk drive, solid state drive, or a collection of storage devices distributed across multiple storage systems, and/or other suitable forms of digital storage, local or remote. The support circuits are conventionally coupled to processor 414 and comprises cache, clock circuits, input/output subsystems, power supplied, and the like, and combinations thereof. I/O device interface 407 allows for the connection of various I/O devices (e.g., keyboards, displays, mouse devices, pen input, etc.) to system 400.

Processor 414 may retrieve and execute programming instructions stored in memory 412. Similarly, processor 414 may retrieve and store application data residing in memory 412. Software routines (programs) and data may be coded and stored within memory 412 for instructing processor 414. Software routines, when executed by processor 414, transform processor 414 into a specific purpose computer (controller) that controls system 100. A software program (or non-transitory computer readable instructions) readable by processor 414 determines which tasks are performable by various components of system 100. Controller 401 may be used to control various components and parameters of system 100 including, for example, OCT unit 102 and/or LJDG 440.

Memory 412 may include non-transitory computer readable instructions corresponding to input parameters 415, OCT/LJDG controller 417 and cornea measurement module 419. Input parameters may be used to control the OCT unit 102 (e.g., OCT interferometer 104 settings) and/or operating settings for LJDG 440. In some embodiments, input parameters 415 may include a speed and volume of liquid drops projected by LJDG 440 and operating parameters of the OCT unit 102 (e.g., laser scan pattern, scan speed, spot size, intensity, pulse duration, and the like). Cornea measurement module 419 may store viscos-elastic response information of the eye and optical response from change of retro-scattering properties of cornea and shape under mechanical stress. OCT/LJDG controller 417 may store OCT mechanical shear wave propagation analysis operations by mean phase-sensitive techniques. Cornea measurement module 419 may include mappings that correlate various conditions of the eye with IOP and Young's modulus values and algorithms for characterization of the biomechanical parameters of the cornea, Young's modulus, and IOP. Controller 401 may determine a diagnosis or provide medical insight based on the biomechanical properties of the cornea and IOP. User interface 402 may output diagnostic information to the clinician.

The detailed description and the drawings are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims.

Furthermore, the embodiments shown in the drawings or the characteristics of various embodiments mentioned in the present description are not necessarily to be understood as embodiments independent of each other. Rather, it is possible that each of the characteristics described in one of the examples of an embodiment may be combined with one or a plurality of other desired characteristics from other embodiments, resulting in other embodiments not described in words or by reference to the drawings. Accordingly, such other embodiments may fall within the scope of the appended claims.

### EXAMPLE EMBODIMENTS

### Embodiment 1

An apparatus for projecting droplets onto a cornea of a patient's eye for ophthalmic diagnostics, the apparatus comprising: a liquid sampling unit configured to project a quantity of liquid; an electric droplet generator configured to receive the quantity of liquid and output one or more electrically charged droplets; a steering unit configured to electrostatically steer the one or more electrically charged droplets along a trajectory onto the cornea of the patient's eye; and a controller, the controller including a processor in communication with a memory having non-transitory machine readable instructions, wherein when executed by the processor, the non-transitory machine readable instructions cause the apparatus to: measure, utilizing an optical coherence tomography (OCT) measurement unit, a response to an impact of the one or more electrically charged droplets on the cornea; determine one or more physical parameters associated with the cornea based on measuring the response; determine, based on the one or more physical parameters, a condition of the cornea; and output, a diagnosis based on the condition of the cornea.

### Embodiment 2

The apparatus of Embodiment 11, wherein the non-transitory machine readable instructions further cause the apparatus to: prior to the impact of the one or more electrically charged droplets on the cornea, sterilize one or more liquid droplets or the one or more electrically charged droplets by emitting one or more beams of ultra violet light across a pathway of the one or more liquid droplets or the one or more electrically charged droplets.

What is claimed is:

1. An apparatus for projecting droplets onto a cornea of a patient's eye for ophthalmic diagnostics, the apparatus comprising:
a liquid sampling unit configured to project a quantity of liquid;
an electric droplet generator configured to receive the quantity of liquid and output one or more electrically charged droplets; and
a steering unit configured to electrostatically steer the one or more electrically charged droplets along a trajectory onto the cornea of the patient's eye.

2. The apparatus of claim 1, wherein the liquid sampling unit comprises:
one or more pressurized containers having an outlet and configured to store liquid; and
a valve coupled to the outlet via one or more conduits,
wherein outputting the quantity of liquid comprises actuating the valve and outputting the quantity of liquid provided by the one or more pressurized containers via the valve.

3. The apparatus of claim 1, wherein the liquid sampling unit comprises:
one or more vented containers having a first outlet and configured to store liquid;
a valve coupled to the first outlet via one or more conduits;
a pump coupled the valve and configured to output the quantity of liquid; and
a pressure sensor controller coupled to the pump, the pressure sensor controller being configured to sense a pressure of the quantity of liquid and adjust the pump based on the pressure of the quantity of liquid.

4. The apparatus of claim 1, wherein the electric droplet generator comprises:
a piezo-electric actuator configured to oscillate a width of a pathway between a first width and a second width; and
a capacitive electrostatic charging unit configured to emit an electric field across the pathway,
wherein the quantity of liquid forms into one or more individual droplets when the pathway oscillates from the first width to the second width, and wherein the one or more individual droplets are charged by the electric field thereby forming the one or more electrically charged droplets.

5. The apparatus of claim 4, wherein the electric droplet generator comprises an ultraviolet light emitter configured to project one or more beams of ultraviolet light across the pathway for sterilizing the one or more electrically charged droplets.

6. The apparatus of claim 1, wherein the steering unit comprises one or more electrodes having one or more electrostatic plates inducing an electric field across a pathway of the electrically charged droplets, wherein the steering unit is configured to alter a strength and a direction of the electric field to control the trajectory of the one or more electrically charged droplets.

7. The apparatus of claim 1, further comprising:
a liquid waste collector coupled to a waste container, wherein the liquid waste collector is configured to trap one or more errant droplets, the one or more errant droplets corresponding to electrically charged droplets that fall outside of the trajectory.

8. The apparatus of claim 7, wherein the waste container is removably coupled to the liquid waste collector.

9. The apparatus of claim 1, further comprising a controller, wherein the controller is in communication with the liquid sampling unit, the electric droplet generator, the steering unit and an optical coherence tomography (OCT) measurement unit.

10. The apparatus of claim 9, wherein the controller is configured to measure a physical state of the cornea utilizing the OCT measurement unit.

11. A method for ophthalmic diagnosis of a physical state of a cornea of a patient's eye, the method comprising:
projecting one or more liquid droplets formed from a quantity of liquid;
electrically charging the one or more liquid droplets thereby forming one or more electrically charged droplets;
steering, utilizing a dynamic electric field, the one or more electrically charged droplets along a trajectory onto the cornea of the patient's eye;
measuring, utilizing an optical coherence tomography (OCT) measurement unit, a response to an impact of the one or more electrically charged droplets on the cornea;
determining one or more physical parameters associated with the cornea based on measuring the response;
determining, based on the one or more physical parameters, a condition of the cornea; and
outputting, a diagnosis based on the condition of the cornea.

12. The method of claim 11, further comprising, prior to the impact of the one or more electrically charged droplets on the cornea,
sterilizing the one or more liquid droplets or the one or more electrically charged droplets by emitting one or more beams of ultraviolet light across a pathway of the one or more liquid droplets or the one or more electrically charged droplets.

13. The method of claim 11, wherein the one or more physical parameters include a response time, a natural frequency, a wave velocity and/or wave attenuation.

14. The method of claim 11, wherein the condition of the patient's eye corresponds to a Young's modulus of the cornea, a topology of the cornea, and/or an intraocular pressure of the cornea.

15. The method of claim 11, wherein the diagnosis comprises cataract treatment based on surgically induced astigmatism, cataract refractive treatment, cataract diffractive treatment, corneal refractive treatment, a risk level of post-LASIK complications, and/or orthokeratology treatment.

16. The method of claim 11, wherein the OCT measuring unit comprises:
an OCT interferometer configured to emit a beam of laser light;
one or more optical components configured to condition the beam of laser light and project the beam of laser light onto the cornea of the patient; and
an eye-tracking unit, configured to track a position of the pupil and detect physical changes of the cornea during the impact of the one or more electrically charged droplets based on the light reflected off the cornea,
wherein the one or more physical parameters are determined based on the physical changes of the cornea.

17. The method of claim 16, wherein the one or more optical components include: a fast-scanning mirror, a slow-scanning mirror, a hot mirror, one or more light emitting diodes, and/or an output lens.

18. The method of claim 11, wherein the quantity of liquid is output by a liquid sampling unit, the liquid sampling unit comprising:
one or more pressurized containers having an outlet and configured to store liquid; and
a valve coupled to the outlet via one or more conduits,
wherein outputting the quantity of liquid comprises actuating the valve and outputting the quantity of liquid via the valve.

19. The method of claim 11, wherein the quantity of liquid is output by a liquid sampling unit, the liquid sampling unit comprising:
one or more vented containers having a first outlet and configured to store liquid;
a valve coupled to the first outlet via one or more conduits;
a pump coupled the valve and configured to output the quantity of liquid; and
a pressure sensor controller coupled to the pump, the pressure sensor controller being configured to sense a pressure of the quantity of liquid and adjust the pump based on the pressure of the quantity of liquid.

20. The method of claim 11, wherein the one or more electrically charged droplets are output by an electric droplet generator, the electric droplet generator comprising:
a piezo-electric actuator configured to oscillate a width of a pathway between a first width and a second width; and
a capacitive electrostatic charging unit configured to emit an electric field across the pathway,
wherein forming the one or more liquid droplets from the quantity of liquid comprises oscillating, utilizing the piezo electric actuator, the width of the pathway from the first width to the second width, and wherein forming the one or more electrically charged droplets comprises charging the one or more liquid droplets utilizing the capacitive electrostatic charging unit.
